(19) United States
(12) Patent Application Publication
(10) Pub. No.: US 2011/0288618 A1
(43) Pub. Date: Nov. 24, 2011

(54) NEUROSTIMULATION LEAD ANCHORS

(75) Inventors: Kevin Alan Glen, Wilton, CA (US); Douglas Michael Ackermann, Palo Alto, CA (US); Roger Chen, Valencia, CA (US)

(73) Assignee: Boston Scientific Neuromodulation Corporation, Valencia, CA (US)

(21) Appl. No.: 13/112,038

(22) Filed: May 20, 2011

Related U.S. Application Data
(60) Provisional application No. 61/346,665, filed on May 20, 2010.

(51) Int. Cl. A61N 1/05

(52) U.S. Cl. 607/116

(57) ABSTRACT

In accordance with the present inventions, anchoring devices for a lead (e.g., a neurostimulation lead) placed on solid tissue (e.g., fascia) and methods of anchoring the lead relative to the tissue are provided. Such methods may include inserting the lead into an epidural space and coupling the lead to a neurostimulation.

### RELATED APPLICATION

The present application claims the benefit under 35 U.S.C. §119 to U.S. provisional patent application Ser. No. 61/346,665, filed May 20, 2010. The foregoing application is hereby incorporated by reference into the present application in its entirety.

### FIELD OF THE INVENTION

The present invention relates to tissue stimulation systems, and more particularly, to devices for anchoring implanted neurostimulation leads relative to the surrounding tissue.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations Peripheral Nerve Stimulation (PNS) systems have demonstrated efficacy in the treatment of chronic pain syndromes and incontinence, and a number of additional applications are currently under investigation. Also, Functional Electrical Stimulation (FES) systems such as the Freehand system by NeuroControl (Cleveland, Ohio) have been applied to restore some functionality to paralyzed extremities in spinal cord injury patients.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. Thus, electrical pulses can be delivered from the neurostimulator to the stimulation leads to stimulate the tissue and provide the desired efficacious therapy to the patient. The neurostimulation system may further comprise a handheld patient programmer in the form of a remote control (RC) to remotely instruct the neurostimulator to generate electrical stimulation pulses in accordance with selected stimulation parameters. The RC may, itself, be programmed by a clinician, for example, by using a clinician's programmer (CP), which typically includes a general purpose computer, such as a laptop, with a programming software package installed thereon.

In the context of an SCS procedure, one or more stimulation leads are introduced through the patient's back into the epidural space, such that the electrodes carried by the leads are arranged in a desired pattern and spacing to create an electrode array. One type of commercially available stimulation lead is a percutaneous lead, which comprises a cylindrical body with ring electrodes, and can be introduced into contact with the affected spinal tissue through a Touhy-like needle, which passes through the skin, between the desired vertebrae, and into the epidural space above the dura layer. For unilateral pain, a percutaneous lead is placed on the corresponding lateral side of the spinal cord. For bilateral pain, a percutaneous lead is placed down the midline of the spinal cord, or two or more percutaneous leads are placed down the respective sides of the midline of the spinal cord, and if a third lead is used, down the midline of the special cord. After proper placement of the stimulation leads at the target area of the spinal cord, the leads are anchored in place at an exit site to prevent movement of the stimulation leads.

To facilitate the location of the neurostimulator away from the exit point of the stimulation leads, lead extensions are sometimes used. The stimulation leads, or the lead extensions, are then connected to the IPG, which can then be operated to generate electrical pulses that are delivered, through the electrodes, to the targeted tissue, and in particular, the dorsal column and dorsal root fibers within the spinal cord. The stimulation creates the sensation known as paresthesia, which can be characterized as an alternative sensation that replaces the pain signals sensed by the patient.

In a typical lead anchoring processor, the physician slips a suture sleeve over a lead, and then sutures the sleeve to fascia to fix the lead within the patient. This process, however, is time consuming and difficult for less experienced surgeons or anesthesiologists to effectively do. Thus, lead fixation without the use of a suture is highly desired, since this will eliminate the time and skill necessary to tie the suture. Additionally, depending on the fixation mechanism, it may be possible to make a smaller incision during the lead fixation step. For clinical reasons, the ability to create a smaller incision is highly desirable.

Furthermore, undesirable migration of the implanted lead frequently results from inadequate anchoring. Thus, having more simple, more efficient, and more reliable anchoring systems are desirable in order to reduce the surgical time, reduce patient recovery time, and reduce the frequency and severity of lead migrations.

### SUMMARY OF THE INVENTION

In accordance with the present inventions, anchoring devices for a lead (e.g., a neurostimulation lead or a catheter) placed on solid tissue (e.g., fascia) and methods of anchoring the lead relative to the tissue are provided. Such methods may include inserting the lead into an epidural space and coupling the lead to a neurostimulator. The lead may have a suitable diameter, e.g., in the range of 0.03 inches and 0.07 inches.

In accordance with a first aspect of the present inventions, an anchoring device comprises a pair of lever arms composed of a biocompatible material, each of the lever arms including a tissue grasping portion and a finger portion. The anchoring device further comprises a hinge coupling the lever arms together between the tissue grasping portion and finger portion of the respective lever arms. The anchoring device further comprises a spring biasing mechanism configured for biasing the tissue grasping portions together while biasing the finger portions away from each other to place the tissue grasping portions in a closed state capable of gripping the lead while grasping the solid tissue. In one embodiment, the biasing mechanism is a spring mechanism (e.g., a torsion spring disposed around the hinge) configured for biasing the tissue grasping portions together while biasing the finger portions away from each other. The spring force allows the finger portions to be displaced toward each other when a manual force is applied to the finger portions to place the tissue grasping portions in an open state capable of allowing the tissue grasping portions to receive the lead, and allows the finger portions to be displaced away from each other when the manual force is released from the finger portions to place the tissue grasping portions in a closed state capable of gripping the lead while grasping the solid tissue. In one embodiment, each finger portion of the respective lever arms includes a plurality of teeth that are capable of embedding within the solid tissue when the manual force is released from the finger portions.

In accordance with a second aspect of the present inventions, a method of using the foregoing anchoring device comprises placing the lead relative to the solid tissue, applying the manual force to the finger portions to place the tissue grasping portions in the open state, locating the anchoring device in contact with the solid tissue about the lead, and releasing the manual force from the finger portions to place the tissue grasping portions in the closed state, thereby gripping the lead while grasping the solid tissue. If each finger portion of the respective lever arms includes a plurality of teeth, they can embed within the solid tissue when the tissue grasping portions grasp the solid tissue.

In accordance with a third aspect of the present inventions, an anchoring device comprises a flange composed of a biocompatible, malleable, material, the flange being pre-curved, such it has two opposing sides, a convex outer surface, and a concave inner surface capable of receiving the lead. In one embodiment, the pre-curved flange is a cylindrical segment. In another embodiment, the flange is pre-curved at least 180 degrees. The anchoring device further comprises two rows of teeth respectively extending from the two opposing sides of the flange. In one embodiment, the teeth are pre-curved in the same direction in which the flange is pre-curved. The flange is configured for deforming when a manual compressive force is applied to the convex outer surface adjacent the two opposing sides, thereby gripping the received lead and embedding the teeth within the solid tissue. In one embodiment, the two rows of teeth are configured for respectively meshing with each other when the flange is deformed.

In accordance with a fourth aspect of the present inventions, a method of using the foregoing anchoring device comprises placing the lead relative to the solid tissue, receiving the lead within the pre-curved flange while the anchoring device is located in contact with the solid tissue about the lead, and applying the manual compressive force to the convex outer surface adjacent the two opposing sides of the pre-curved flange, thereby gripping the lead and embedding the teeth within the solid tissue.

In accordance with a fifth aspect of the present inventions, another anchoring device comprises a sleeve having a lumen through which the lead can be secured, and a pair of wings transversely extending in opposite directions from the sleeve. The wings are composed of a material (e.g., a mesh) that can be affixed to the solid tissue to fix the lead relative to the solid tissue. In one embodiment, the wings are composed of a material through which staples can penetrate into the solid tissue.

In accordance with a sixth aspect of the present inventions, another method comprises introducing the lead through a lumen of a sleeve, securing the lead within the lumen of the sleeve, placing the sleeve with the lead secured therein relative to the solid tissue, and stapling the sleeve to the solid tissue to fix the lead relative to the solid tissue. In one method, the sleeve has eyelets, and staples are inserted through the eyelets to staple the sleeve to the solid tissue. In another method, a pair of wings (e.g., composed of a mesh material) extends from the sleeve in opposite directions, and staples are inserted through the wings to staple the sleeve to the solid tissue.

In accordance with a seventh aspect of the present inventions, another anchoring device comprises a sleeve having a lumen through which the lead can be secured, eyelets on opposing sides of the sleeve. In one embodiment, the anchoring device may further comprise a pair of tabs disposed on opposite sides of the sleeve, wherein the eyelets are respectively formed in the tabs. The anchoring device further comprises anchoring elements for respective interaction with the eyelets. Each of the anchoring elements includes a head having a size greater than the respective eyelet and a shaft element configured for being advanced through the respective eyelet to penetrate the solid tissue, thereby fixing the lead relative to the solid tissue.

In one embodiment, each shaft element includes a helical screw configured for penetrating the solid tissue when the respective anchoring element is rotated. In this case, each of the heads may include a recess (e.g., a hexagonal recess) capable of receiving a tool for rotating the respective anchoring element. In another embodiment, each shaft element includes a spike configured for penetrating the solid tissue when the respective anchoring device is pushed. Each shaft element may include at least one barb element located on the respective spike. The barb element(s) may be configured for breaking off of the spike when the respective anchoring element is removed from the solid tissue. Alternatively, each spike may include a sheath and a stem carrying the barb element(s), in which case, the sheath may slidably receive the stem and include at least one aperture from which the barb element(s) may be respectively deployed when the stem is slid proximally relative to the sheath and within which the barb element(s) may be respectively retracted when the stem is slid distally relative to the sheath.

In an optional embodiment, each shaft element includes a plurality of holes along its length configured for promoting tissue in-growth. In another optional embodiment, each anchoring device further includes a retainer through which the respective shaft element extends, the retainer being located on the side of the respective eyelet opposite to the respective head to hold the respective anchoring element within the sleeve

In accordance with an eighth aspect of the present inventions, a method of using the foregoing anchoring device comprises introducing the lead through the lumen of the sleeve, securing the lead within the lumen of the sleeve, and advancing the shaft element of each anchoring element through a respective one of the eyelets to penetrate the solid tissue until the head contacts the respective eyelet, thereby fixing the lead relative to the solid tissue. In the case where each shaft element includes a helical screw, it may be advanced through the respective one of the eyelets to penetrate the solid tissue by rotating the respective anchoring element. In the case where each shaft element includes a spike, it may be advanced through the respective one of the eyelets to penetrate the solid tissue by pushing the respective anchoring element.

If each anchoring element further includes a barb element located on the respective spike, it may resist removal of the anchoring element from the solid tissue. The barb element(s) may break off from the spike by removing the anchoring element from the solid tissue. In the case where each spike includes a sheath and a stem carrying the barb element(s), the stem may slidably receive the stem and include at least one aperture, and the method may comprise sliding the stem proximally relative to the sheath, thereby deploying the at least one barb element from the at least one aperture into the solid tissue, and sliding the stem distally relative to the sheath, thereby retracting the barb element(s) within the aperture(s).

In accordance with a ninth aspect of the present inventions, an anchoring device comprises a planar substrate composed of a biocompatible material. The planar substrate may be flexible and may have a thickness in the range of 0.01-0.1 in. The anchoring device further comprises a plurality of barb elements extending from one surface of the planar substrate. The barb elements configured for penetrating and adhering to the solid tissue when the planar substrate is pressed over solid tissue, thereby fixing the lead relative to the solid tissue. In one embodiment, the lengths of the barb elements are longer than the diameter of the lead, such that the barb elements can penetrate and adhere to the solid tissue when the planar substrate is pressed over the lead and solid tissue. In another embodiment, the anchoring device further comprises a sleeve coupled to another surface of the planar substrate opposite to the one surface, the sleeve having a lumen through which the lead can be secured.

In accordance with a tenth aspect of the present inventions, a method of using the foregoing anchoring device comprises placing the lead relative to the solid tissue, and pressing the planar substrate against the solid tissue to adhere the barb elements to the solid tissue, thereby fixing the lead relative to the solid tissue. In one method, the planar substrate is pressed against the lead and solid tissue to fixe the lead relative to the solid tissue. Another method further comprises introducing the lead through a lumen of a sleeve coupled to the another surface of the planar substrate opposite to the one surface, and securing the lead within the lumen of the sleeve.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of a SCS system in use with a patient;

FIG. 2 is a perspective view of one embodiment of an anchoring device that can be used to fix the lead in the SCS system of FIG. 1 relative to tissue;

FIG. 3 is a profile view of the anchoring device of FIG. 2 in an open state;

FIG. 4 is a profile view of the anchoring device of FIG. 2 in a closed state;

FIGS. 5a-5d are plan views illustrating one method of using the anchoring device of FIG. 2 to affix the lead in the SCS system of FIG. 1 relative to tissue;

FIG. 6 is a perspective view of another embodiment of an anchoring device that can be used to fix the lead in the SCS system of FIG. 1 relative to tissue, particularly shown in an open state;

FIG. 7 is a perspective view of the anchoring device of FIG. 6 in a closed state;

FIGS. 8a-8b are plan views illustrating one method of using the anchoring device of FIG. 6 to affix the lead in the SCS system of FIG. 1 relative to tissue;

FIG. 9 is a perspective view of still another embodiment of an anchoring device that can be used to fix the lead in the SCS system of FIG. 1 relative to tissue;

FIGS. 10a-10e are plan views illustrating a method of using the anchoring device of FIG. 9 as well as other methods used to affix the lead in the SCS system of FIG. 1 relative to tissue;

FIG. 11 is a perspective view of yet another embodiment of an anchoring device that can be used to fix the lead in the SCS system of FIG. 1 relative to tissue;

FIG. 12 is a perspective view of yet another embodiment of an anchoring device that can be used to fix the lead in the SCS system of FIG. 1 relative to tissue;

FIGS. 13a-13e are perspective view of different barb elements that can be used in the anchoring device of FIG. 12;

FIGS. 14a-14b are plan views of one embodiment of a spike that can be used in the anchoring device of FIG. 12;

FIG. 15 is a perspective view of an optional embodiment of a spike that can be used in the anchoring device of FIG. 12;

FIG. 16 is an alternative embodiment of the anchoring device of FIG. 12;

FIGS. 17a-17d are plan views illustrating a method of using the anchoring devices of FIGS. 11 and 12 to affix the lead in the SCS system of FIG. 1 relative to tissue;

FIG. 18 is a perspective view of yet another embodiment of an anchoring device that can be used to fix the lead in the SCS system of FIG. 1 relative to tissue;

FIG. 19 is a perspective view of yet another embodiment of an anchoring device that can be used to fix the lead in the SCS system of FIG. 1 relative to tissue;

FIGS. 20a-20c are plan views illustrating a method of using the anchoring devices of FIGS. 18 and 19 to affix the lead in the SCS system of FIG. 1 relative to tissue.

### DETAILED DESCRIPTION OF THE EMBODIMENTS

At the outset, it should be noted that the present invention is directed to the fixation of implantable leads, such as neural stimulation leads or cardiac leads, and more particularly to the fixation of electrodes or electrode arrays attached to the neural stimulation leads or cardiac leads, so that such electrodes or electrode arrays remain in a desired position relative to the tissue that is to be stimulated. It should further be noted that the principles and teachings of the invention may be used with any kind of neural stimulation lead, cardiac lead, or catheter, particularly those that are implanted within a tissue cavity (the presence of which cavity allows undesirable movement of the lead). For the purposes of this specification, a “lead” will be considered as any elongated medical device designed to be implanted and affixed within a patient's body. Thus, while the invention will be described in terms of a spinal cord stimulation (SCS) lead adapted for implantation in the epidural space next to the spine, it is to be understood that such description is intended to be only exemplary and not limiting unless otherwise defined as such.

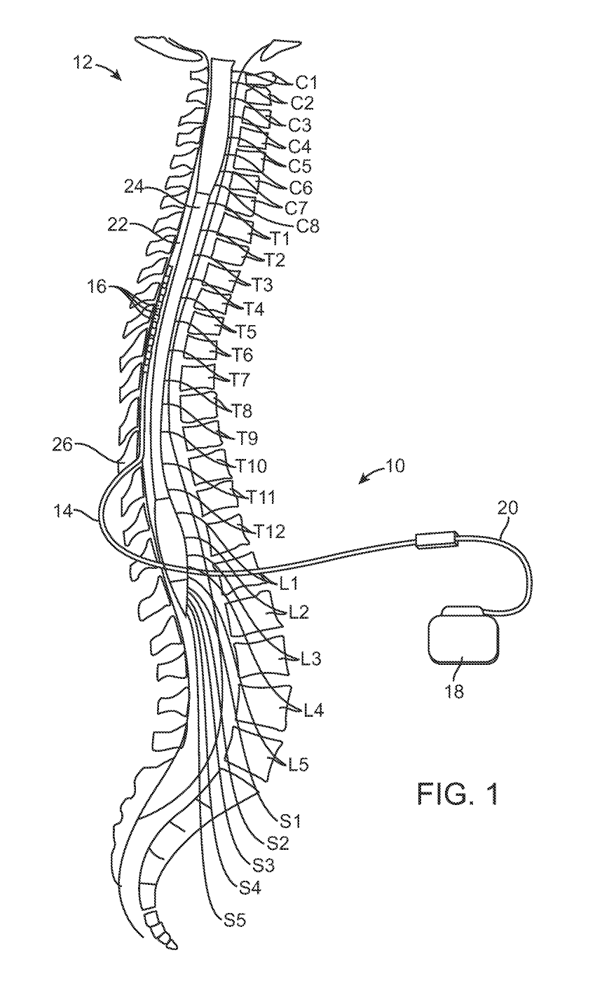

Turning first to FIG. 1, an exemplary SCS system 10 is shown implanted in a patient 12. The system 10 generally includes an implantable percutaneous neurostimulation lead 14 carrying a distal array of electrodes 16, an implantable pulse generator (IPG) 18, and an optional percutaneous lead extension 20 used to connect the neurostimulation lead 14 to the IPG 18. The neurostimulation lead 14, IPG 18, and lead extension 20 may be coupled to each other using suitable connectors, such as those disclosed in U.S. Pat. Nos. 6,609,029 and 6,741,892, which are expressly incorporated herein by reference. The preferred placement of the neurostimulation lead 14 is within the epidural space 22 of the patient 12 adjacent the area of the spinal cord 24 to be stimulated. Due to the lack of space near the location where the neurostimulation lead 14 exit the spinal column 26, the IPG 18 is generally implanted in a surgically-made pocket either in the abdomen or above the buttocks. The IPG 18 may, of course, also be implanted in other locations of the patient's body.

The neurostimulation lead 14 may have a diameter of, e.g., between about 0.03 inches to 0.07 inches and a length within the range of 30 cm to 90 cm for spinal cord stimulation applications. The body of the neurostimulation lead 14 may be composed of a suitable electrically insulative material, such as, a polymer (e.g., polyurethane or silicone), and may be extruded from as a unibody construction. The lead extension 20 may be similarly constructed, with the exception that the lead extension 20 does not carry any stimulating electrodes. Notably, for the purposes of this specification, when a lead extension is coupled to a neurostimulation lead, the combination can be considered as a neurostimulation lead. Further details describing the construction and method of manufacturing percutaneous stimulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

The IPG 18 includes a hermetically sealed housing and pulse generation circuitry contained in the housing for delivering electrical stimulation energy in the form of a pulsed electrical waveform (i.e., a temporal series of electrical pulses) to the electrode array 16 in accordance with a set of stimulation parameters. The IPG 18 may be programmed by an external device, such as a remote control (RC). Further details discussing IPG's are described more fully in U.S. Pat. Nos. 6,516,227 and 6,993,384, which are expressly incorporated herein by reference. It should be noted that rather than an IPG, the SCS system 10 may alternatively utilize a different neurostimulator, such as an implantable receiver-stimulator (not shown), connected to the neurostimulation lead 14. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the control signals and generates the stimulation in accordance with these signals.

Once the neurostimulation lead 14 is located within the epidural space 20 in the desired position, which can be verified by operating the IPG 18 using different sets of stimulation parameters in a conventional manner until optimal or otherwise effective paresthesia is felt by the patient, the neurostimulation lead 14 may be affixed relative to the tissue surrounding the neurostimulation lead 14, which in the illustrated embodiment, may be accomplished by affixing the portion of the neurostimulation lead 14 exiting the spinal column 26 to the fascia surrounding the back muscle. As will now be described, any one or more of a variety of anchoring devices can be used to efficiently and effectively affix neurostimulation lead 14 to the fascia.

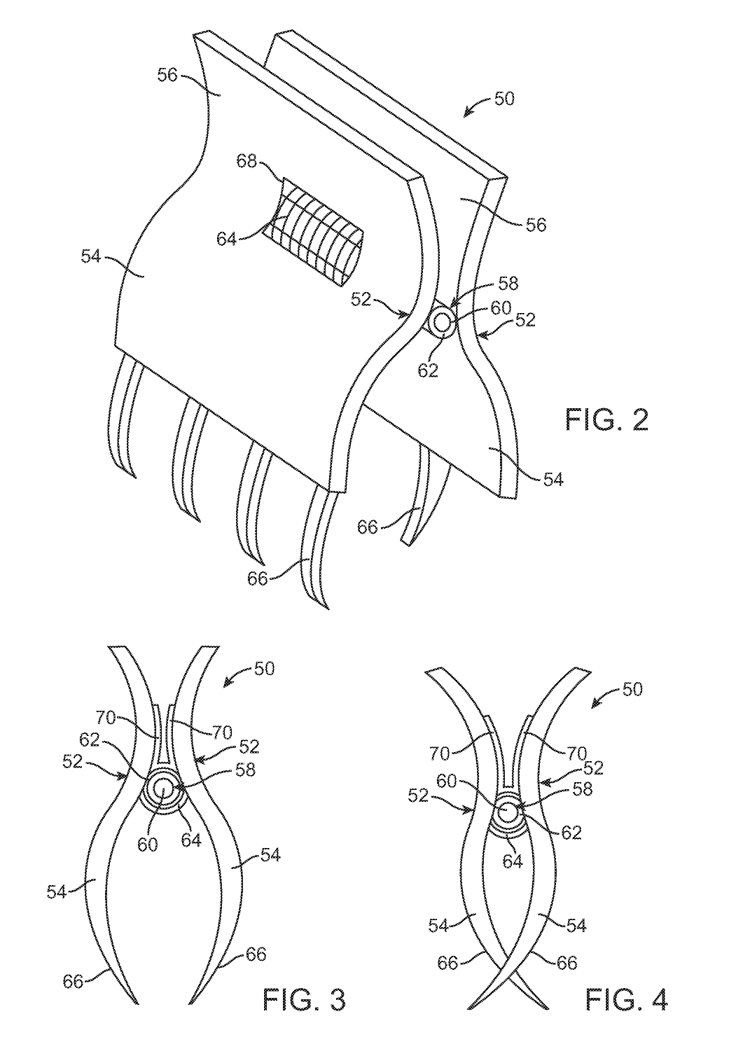

With reference to FIGS. 2-4, one embodiment of an anchoring device 50 will now be described. The anchoring device 50 comprises a pair of lever arms 52, each of which includes a tissue grasping portion 54 and a finger portion 56. The lever arms 52 are composed of a suitable rigid biocompatible material, such as, titanium, stainless steel, polycarbonate, etc. The anchoring device 50 further comprises a hinge 58 coupling the lever arms 52 together between the tissue grasping portion 54 and the finger portion 56 of the respective lever arms 52. In the illustrated embodiment, the hinge 58 comprises a pin 60 composed of a suitable biocompatible material, such as stainless steel, and a lumen 62 extending through the lever arms 52 in which the pin 60 is disposed. As such, the lever arms 52 may rotate about the hinge 58 relative to each other, such that the tissue grasping portions 54 can be alternately placed between an open state (FIG. 3) and a closed state (FIG. 4).

The anchoring device 50 further comprises a biasing mechanism, such as a spring mechanism 64 composed of a suitable resilient biocompatible material (e.g., stainless steel). The spring mechanism 64 is configured for biasing the tissue grasping portions 54 together while biasing the finger portions 56 away from each other. This spring force allows the finger portions 56 to be displaced toward each other when a manual force is applied to the finger portions 56 to place the tissue grasping portions 54 in an open state capable of allowing the tissue grasping portions 54 to receive the neurostimulation lead 14, and allowing the finger portions 56 to be displaced away from each other when the manual force is released from the finger portions 54 to place the tissue grasping portions 54 in a closed state capable of gripping the neurostimulation lead 14 while grasping the solid tissue.

Preferably, each finger portion 56 of the respective lever arms 52 includes a plurality of teeth 66 that are capable of embedding within the solid tissue when the manual force is released from the finger portions 56. The teeth 66 may optionally have barbs (not shown) that prevent the teeth 66 from dislodging from the tissue. In the illustrated embodiment, the spring mechanism 64 takes the form of a helical spring, and in particular a torsion spring, disposed around the hinge 58. In this case, cutouts 68 (only one shown in FIG. 2) are provided in the respective lever arms 52 to spatially accommodate the torsion spring 64. As illustrated in FIGS. 3 and 4, the ends of the torsion spring 64 are formed into legs 70 that urge the opposing inner surfaces of the finger portions 56 away from each other to bias the tissue grasping portions 54 into the closed state.

Although the spring mechanism 64 is illustrated and described as a coiled spring, it should be noted that any mechanism that can store a spring force, including a non-coiled mechanism, can be used as the spring mechanism 64. Alternatively, the biasing mechanism may take the form of an element other than a spring mechanism, such as a set screw (not shown). In this case, the set screw can be disposed through a thread located on one finger portion 56, such that the proximal end of the set screw (presumably, having a recess such as a slot for receiving a tool) is exposed on the outer surface of the lever arms 52, and a distal end that contacts the inner surface of the other finger portion 56. In this manner, the set screw can be rotated to provide a biasing force between the respective lever arms 52, thereby displacing the finger portions 56 away from each other while displacing the tissue grasping portions toward each other to place the tissue grasping portions 54 in a closed state capable of gripping the neurostimulation lead 14 while grasping the solid tissue.

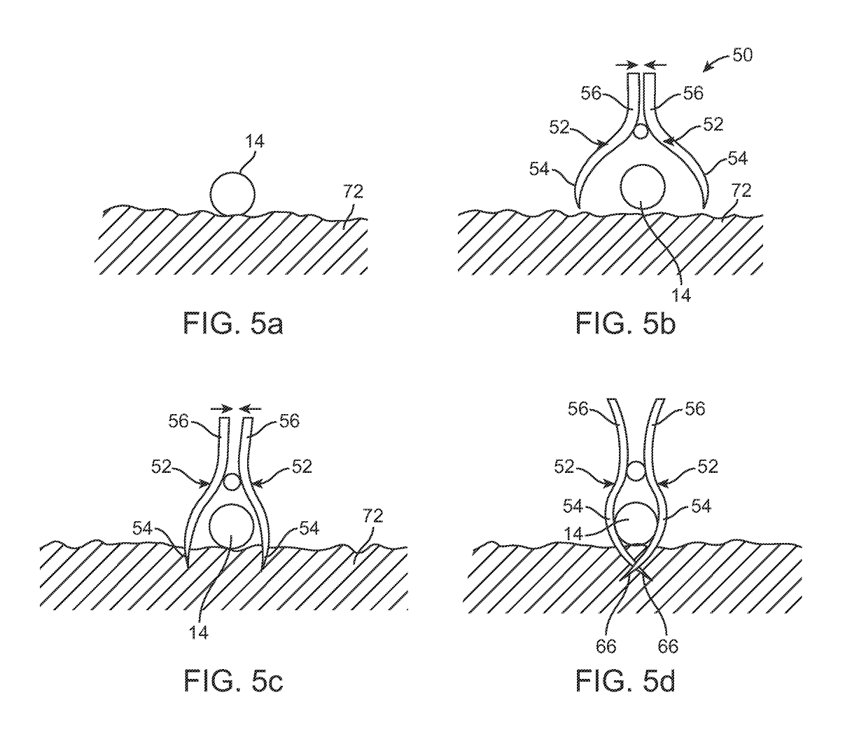

Having described the structure of the anchoring device 50, one exemplary method of fixing the neurostimulation lead 14 relative to solid tissue 72 using the anchoring device 50 will now be described with respect to FIGS. 5a-5d. First, the neurostimulation lead is placed relative to the tissue 72 (FIG. 5a), after which a manual force (in direction of arrows) is applied to the finger portions 56 to place the tissue grasping portions 54 in the open state (FIG. 5b). Next, the anchoring device 50 is located in contact with, and in particular partially embedded within, the tissue 72 (FIG. 5c), and then the manual force is released from the finger portions 56 to place the tissue grasping portions 54 in the closed state, thereby gripping the neurostimulation lead 14 while grasping the tissue 72 (FIG. 5d). In the advantageous case where the lever arms 52 include teeth 66, the teeth 66 are embedded within the tissue 72 when the tissue grasping portions 58 grasp the tissue 72.

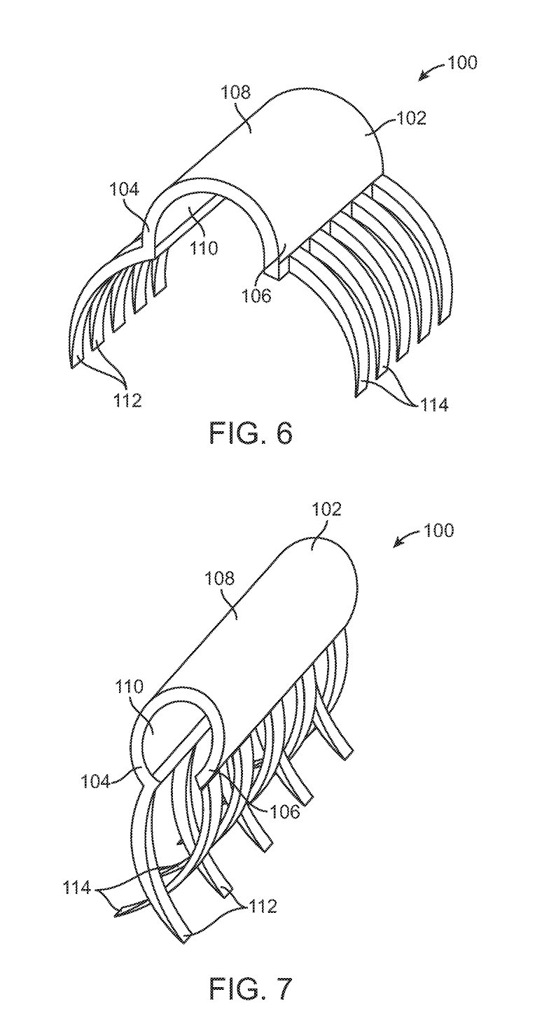

With reference to FIGS. 6 and 7, another embodiment of an anchoring device 100 will now be described. The anchoring device 100 comprises a flange 102 composed of a biocompatible, malleable, material, such as, e.g., Titanium or stainless steel 316L. The flange 102 is pre-curved, such it has two opposing sides 104, 106, a convex outer surface 108, and a concave inner surface 110 capable of receiving the neurostimulation lead 14. In the illustrated embodiment, the flange 102 is a cylindrical segment that preferably has an angular range of 150-210 degrees. The flange 102 is shown in FIG. 6 as being a 180 degree cylindrical segment.

The anchoring device 100 further comprises two rows of teeth 112, 114 respectively extending from the two opposing sides 104, 106 of the flange 102. Significantly, the flange 102 is configured for deforming when a manual compressive force is applied to the convex outer surface 108 adjacent the two opposing sides 104, 106, thereby gripping the received lead 14 and embedding the teeth 112, 114 within the tissue. In the preferred embodiment, teeth 112, 114 are pre-curved in the same direction in which the flange 102 is pre-curved, such that the teeth 112, 114 more easily embed into the tissue as the flange 102 is deformed. The two rows of teeth 112, 114 are configured for respectively meshing with each other when the flange 102 is deformed.

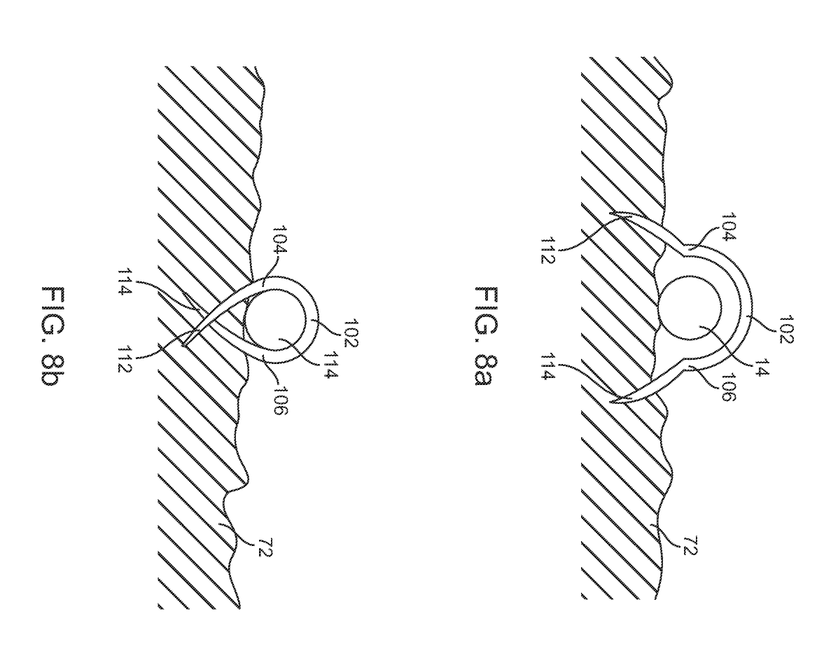

Having described the structure of the anchoring device 100, another exemplary method of fixing the neurostimulation lead 14 relative to solid tissue 72 using the anchoring device 100 will now be described with respect to FIGS. 8a-8b. After the neurostimulation lead 14 is placed relative to the tissue 72 in the manner illustrated in FIG. 5a, the neurostimulation lead 14 is received within the pre-curved flange 102 as the anchoring device 100 is placed in contact with, and in particular embedded within, the tissue 72 (FIG. 8a), and the manual compressive force is applied to the convex outer surface 108 adjacent the two opposing sides 104, 106 (e.g., using a pair of surgical clamps (not shown)), thereby gripping the neurostimulation lead 14 while the teeth 112, 114 mesh together to grasp the tissue 72 (FIG. 8b).

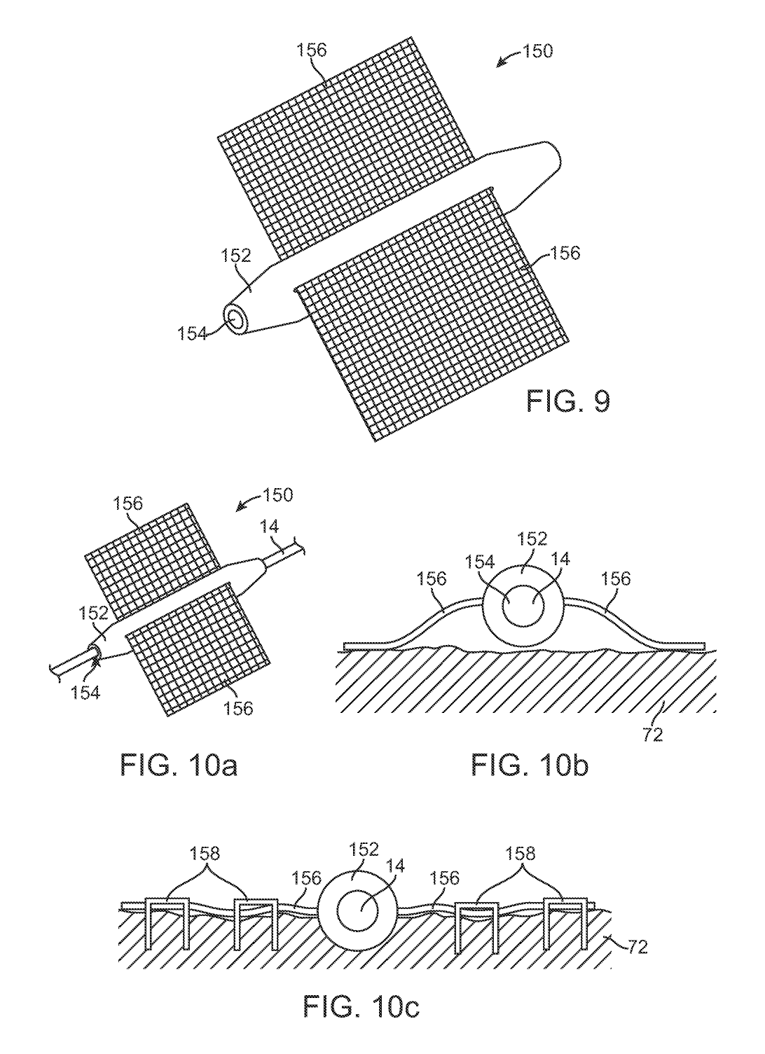

With reference to FIG. 9, another embodiment of an anchoring device 150 will now be described. The anchoring device 150 comprises a sleeve 152 having a lumen 154 through which the neurostimulation lead 14 can be secured. The sleeve 152 is preferably made of a molded, soft polyurethane, such as Tecothane® polyether-based thermoplastic polyurethane (available from Thermedics, Inc., Woburn, Mass.), and most preferably 85A Tecothane® material. Other biocompatible materials, such as epoxy or silicone, may be used, as will be recognized by those of skill in the art. In one embodiment, the diameter of the lumen 154 is slightly less than the diameter of the neurostimulation lead 14, such that the sleeve 152 frictionally secures the neurostimulation lead 14 when introduced through the lumen 154. In an alternative embodiment, a spring (not shown) can be disposed within the lumen 154 for gripping the neurostimulation lead 14, as disclosed in U.S. Pat. No. 6,473,654, which is expressly incorporated herein by reference.

The anchoring device 150 further comprises a pair of wings 156 transversely extending in opposite directions from the sleeve 152. The wings 156 are composed of a material through which staples can penetrate into the solid tissue to fix the neurostimulation lead 14 relative to the solid tissue. Alternatively, the wings 156 are composed of a material that can be bonded to solid tissue with a biocompatible glue. In the preferred embodiment, the wings 156 are composed of a mesh composed of a suitable material, such as woven Dacron®. The mesh wings 156 may be coupled to the sleeve 152 using any one of a variety of manners. For example, the sleeve 152 may be sandwiched between two mesh layers that are bonded together to for the two wings 156, or the sleeve 152 may be molded with the two mesh wings 156.

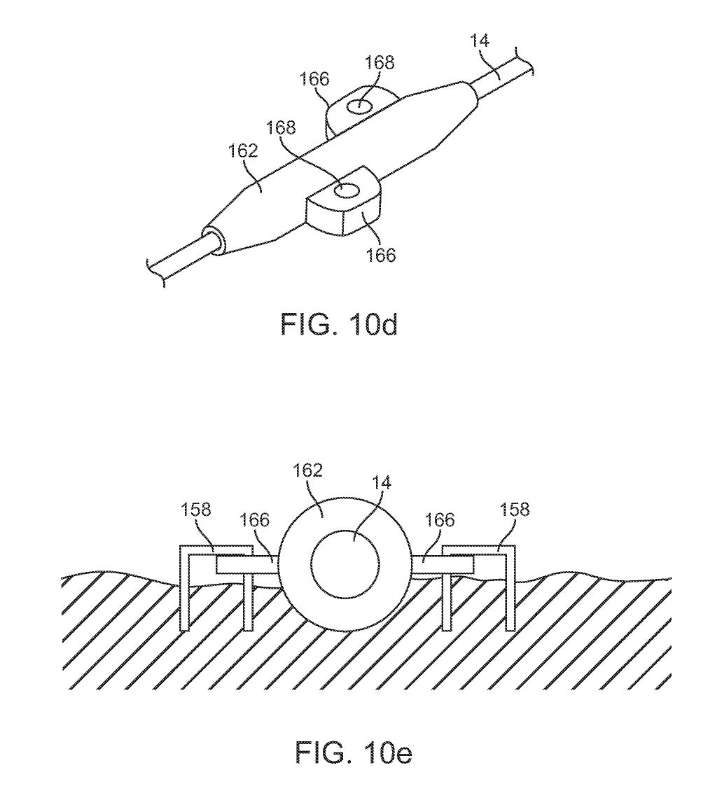

Having described the structure of the anchoring device 150, another exemplary method of fixing the neurostimulation lead 14 relative to solid tissue 72 using the anchoring device 150 will now be described with respect to FIGS. 10a-10c. First, the neurostimulation lead 14 is introduced through and secured within the lumen 154 of the sleeve 152 in a conventional manner (FIG. 10a), and the sleeve 152 with the neurostimulation lead 14 secured therein is located relative to the tissue 72 (FIG. 10b). Next, the sleeve 152 is stapled to the tissue 72 to fix the neurostimulation lead 14 relative to the tissue 72 by inserting staples 158 through the wings 156 into the tissue 72 using a conventional staple gun (not shown) (FIG. 10c). Alternatively, the wings 156 can be bonded to the tissue 72 to fix the neurostimulation lead 14 relative to the tissue 72.

In an alternative method illustrated in FIGS. 10d-10e, the neurostimulation lead 14 is introduced through and secured within the lumen 164 of a conventional sleeve 162 that includes a pair of tabs 166 with eyelets 168 (FIG. 10d). The sleeve 162 may then be stapled to the tissue 72 to fix the neurostimulation lead 14 relative to the tissue 72 by inserting staples 158 through the eyelets 168 into the tissue 72 using a conventional staple gun (not shown) (FIG. 10e).

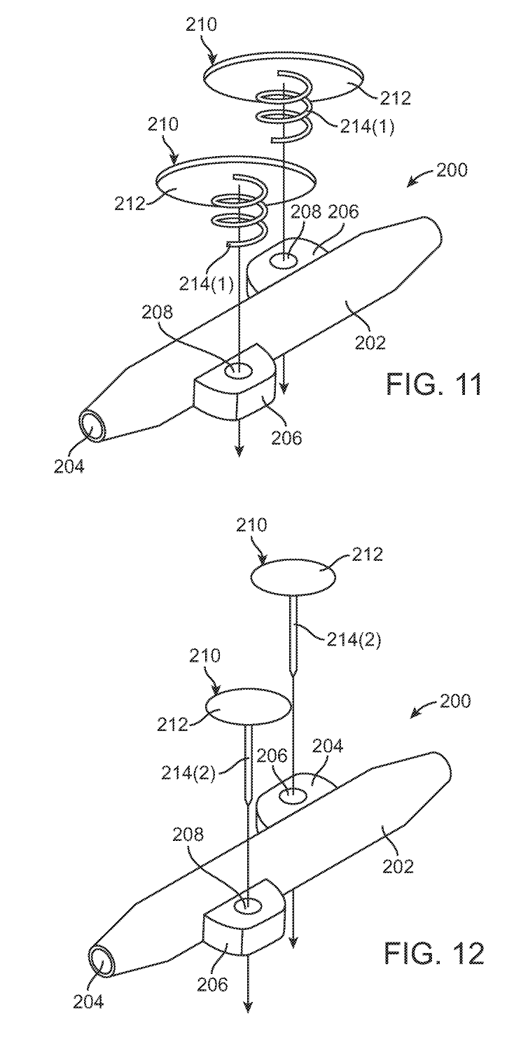

With reference to FIGS. 11-16, another embodiment of an anchoring device 200 will now be described. The anchoring device 200 comprises a sleeve 202 having a lumen 204 through which the neurostimulation lead 14 can be secured. The sleeve 202 can be composed as the same material as sleeve 152 discussed above, and the neurostimulation lead 14 can be secured within the lumen 204 of the neurostimulation lead 14 in the same manner as it can be secured within the lumen 154 of the sleeve 152 discussed above. The anchoring device 200 further comprises a pair of tabs 206 transversely disposed on opposite sides of the sleeve 202, and a pair of eyelets 208 disposed through the respective tabs 206. In the illustrated embodiment, only two tabs 206 with corresponding eyelets 208 are shown, although more than two (e.g., four) sets of tabs 206 and corresponding eyelets 208 can be used.

The anchoring device 200 further comprises a plurality of anchoring elements 210 for respective interaction with the eyelets 208. The anchoring elements 210 may be composed of a suitable rigid, biocompatible material, such as stainless steel. Each of the anchoring elements 210 includes a head 212 having a size greater than the respective eyelet 208 and a shaft element 214 configured for being advanced through the respective eyelet 208 to penetrate the tissue, thereby fixing the neurostimulation lead 14 relative to the tissue.

In the embodiment illustrated in FIG. 11, each shaft element 214 takes the form of a helical screw 214(1) configured for penetrating the tissue when the respective anchoring element 210 is rotated. In this case, each head 212 is large enough to allow a user to easily grasp and rotate it while the screw 214(1) penetrates the tissue. Alternatively, each head 212 may include an element, such as a recess (e.g., a hexagonal recess), capable of receiving a tool, e.g., a hex wrench (not shown), for rotating the respective anchoring element 210. In an optional embodiment, the anchoring element 210 includes a barb (not shown) located at the distal end of the screw 214(1) to prevent incidental removal of the anchoring element 210.

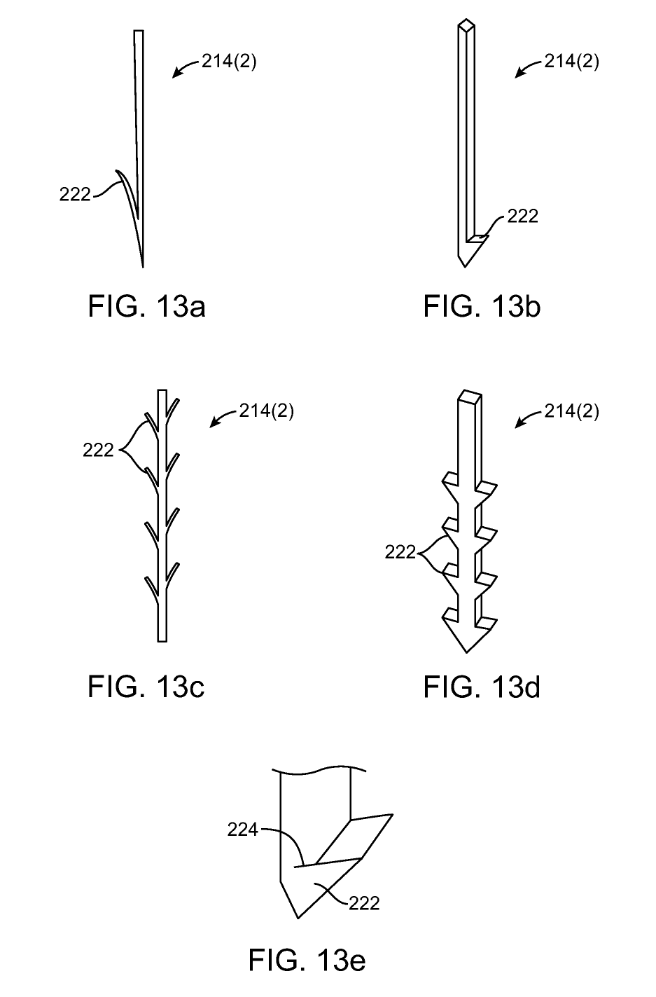

In another embodiment, each shaft element 214 takes the form of a spike 214(2) configured for penetrating the tissue when the respective anchoring element 210 is pushed, as shown in FIG. 12. In this case, each head 212 may be sized to facilitating pushing of the anchoring element with the user's thumb. In some embodiments, the spike 214(2) takes the form of a simple barb in that it includes a single barb element 222 located at the tip of the spike 214(2). The single barb element 222 can, e.g., be shaped as illustrated in FIG. 13a or shaped as illustrated in FIG. 13b. In other embodiments, the spike 214(2) takes the form of a compound barb in that it includes a plurality of barb elements 222 located along the length of the spike 214(2). The barb elements 222 can, e.g., be shaped as illustrated in FIG. 13c or shaped as illustrated in FIG. 13d.

In either case, the barb element or elements 222 should be large enough to facilitate grasping of the tissue by the spike 214(2) while allowing removal of the anchoring element 210 (give sufficient pull-out force) should it be desired to remove the anchoring element 210 at some future time. Alternatively, the barb elements 222 are configured for breaking off when the anchoring elements 210 are removed from the solid tissue. For example, a cut 224 can be formed between a barb element 222 and the spike 214(2), as shown in FIG. 13(e), to facilitate breaking off of the barb element 222 during removal of the anchoring element 210.

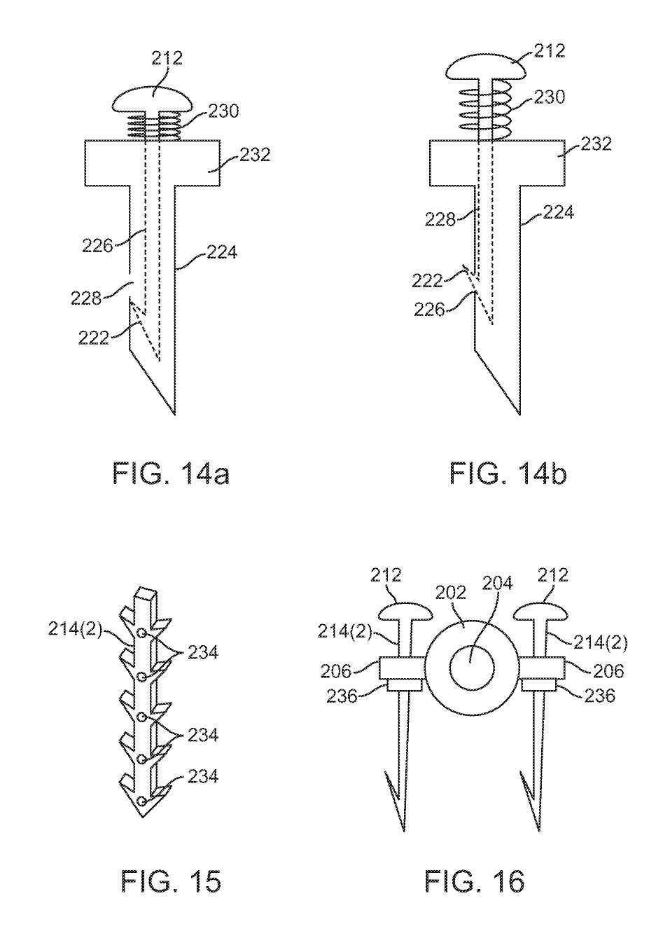

In still another embodiment, each spike 214(2) includes a sheath 224 and a stem 226 carrying at least one of the barb elements 222 (FIGS. 14a and 14b). The sheath 224 slidably receives the stem 226 and includes at least one aperture 228 (one for each barb element 222) from which the barb elements 222 may be respectively deployed when the stem 226 is slid distally relative to the sheath 224 (FIG. 14b) and within which the barb elements 222 may be respectively retracted when the stem 226 is slid proximally relative to the sheath 224 (FIG. 14a). For purposes of brevity, only one aperture 228 and one barb element 222 is shown. In this case, the anchoring element 210 may include a spring 230 located between the head 212, which is formed on the stem 226, and a flange 232 formed on the sheath 224, such that the stem 226 is biased to be in a distal position relative to the sheath 224, thereby biasing the barb element 222 to be deployed from the aperture 228.

In yet another embodiment, each shaft element 214 includes a plurality of holes along its length for promoting tissue in-growth. For example, each spike 214(2) may include a plurality of holes 234 along its length for promoting tissue in-growth, as illustrated in FIG. 15. As a result, the anchoring element 210 can more securely fix the neurostimulation lead 14 relative to the tissue once scarring has occurred. However, the anchoring element 210 can still be removed from the tissue once sufficient force is applied.

In still another embodiment, each anchoring element 210 further includes a retainer through which the respective shaft element 214 extends, with the retainer being located on the side of the respective eyelet 208 of the sleeve 202 opposite to the respective head 212 to hold the respective anchoring element 210 within the eyelet 208. For example, the spike 214(2) can extend through the retainer 236, with the retainer 236 being located on the side of the respective eyelet 208 opposite to the respective head 212, as illustrated in FIG. 16. As a result, the sleeve 202 and anchoring elements 210 can be integrated together into a single device for ease of use.

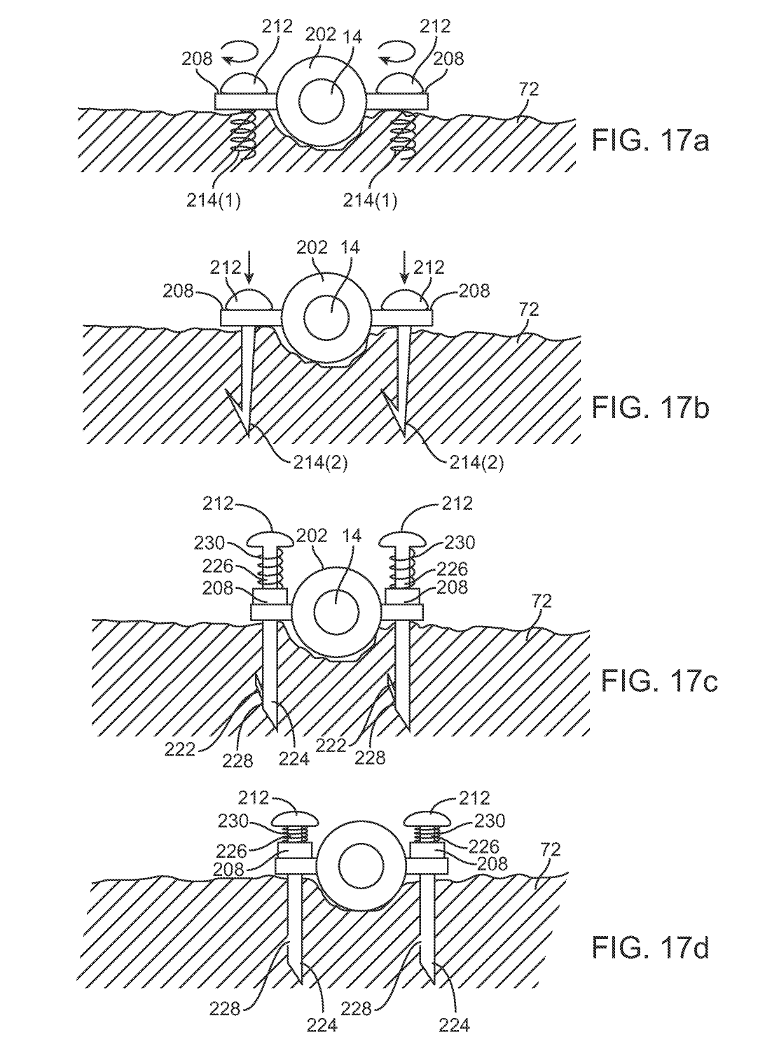

Having described the structure of the anchoring device 200, another exemplary method of fixing the neurostimulation lead 14 relative to solid tissue 72 using the anchoring device 200 will now be described with respect to FIGS. 17a-17d. First, the neurostimulation lead 14 is introduced through and secured within the lumen 204 of the sleeve 202, and the sleeve 202 with the neurostimulation lead 14 secured therein is located relative to the tissue 72 in a similar manner as the neurostimulation lead 14 is secured within the lumen of the sleeve 162 and located relative to the tissue 72 illustrated in FIGS. 10a-10b. Next, the shaft element 214 of each anchoring element 210 is advanced through a respective one of the eyelets 208 to penetrate the tissue 72 until the head 212 contacts the respective eyelet 208, thereby fixing the neurostimulation lead 14 relative to the tissue 72.

In the case where the shaft element takes the form of the helical screw 214(1), it may be advanced through the respective one of the eyelets 208 to penetrate the tissue by rotating (shown by arrow) the respective anchoring element 210 (FIG. 17a). In the case where the shaft element takes the form of a spike 214(2), it is advanced through the respective one of the eyelets 208 to penetrate the tissue 72 by pushing (shown by arrow) the respective anchoring element 210 (FIG. 17b). In the case where each spike 214(2) includes a sheath 224 and a stem 226 slidably received within the sheath 224, the head 212 of each anchoring element 210 can be released to allow the spring 230 to displace the stem 226 proximally relative to the sheath 224, thereby deploying the barb element 222 from the aperture 228 into the tissue 72 (FIG. 17c).

An appropriate time, each anchoring element 210 may be removed from the tissue 72, so that, e.g., the neurostimulation lead 14 may be readjusted or removed altogether from the patient. In the case where the shaft element takes the form of a spike 214(2), the barb element or barb elements 222 may break off into the tissue when the respective spike 214(2) is removed. Alternatively, in the case where each spike 214(2) includes a sheath 224 and a stem 226 slidably received within the sheath 224, each spike 214(2) can be pushed down against the biasing effect of the spring 230 to displace the stem 226 distally relative to the sheath 224, thereby retracting the barb element 22 from the tissue 72 into the aperture 228 into the tissue 72 (FIG. 17d). With the barb element 222 retracted inside of the spike 214(2), the spike 214(2) can then be more easily removed from the tissue 72.

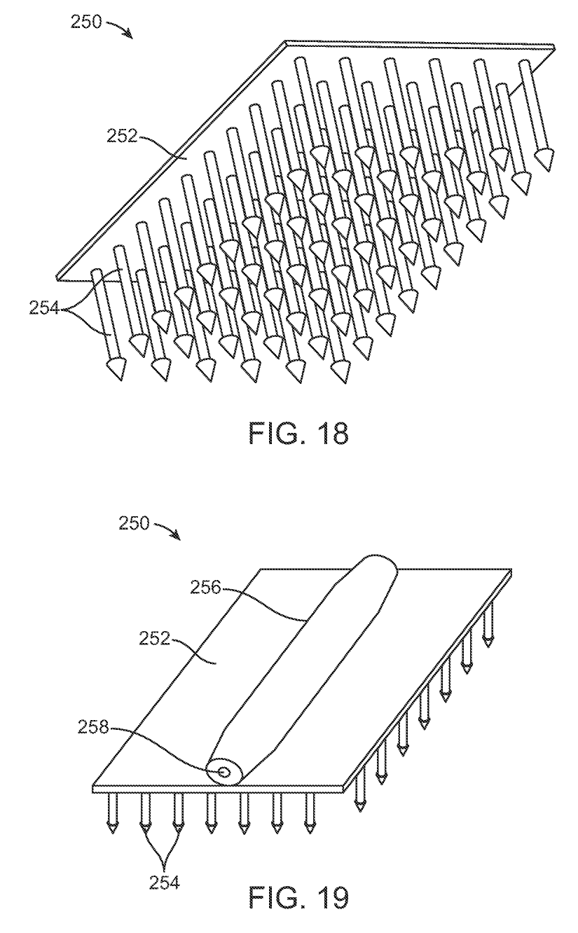

With reference to FIG. 18, still another embodiment of an anchoring device 250 will now be described. The anchoring device 250 comprises a planar substrate 252 composed of a biocompatible, material, e.g., soft polyurethane, such as Tecothane® polyether-based thermoplastic polyurethane (available from Thermedics, Inc., Woburn, Mass.), and most preferably 85A Tecothane® material. Other biocompatible materials, such as epoxy or silicone, may be used, as will be recognized by those of skill in the art. The thickness of the planar substrate 252 may be in a range that allows the substrate 252 to be flexible yet durable, e.g., 0.01-0.10 in. The anchoring device 250 further comprises a plurality of barb elements 254 extending from one surface of the planar substrate. The barb elements 254 are composed of a suitable rigid, biocompatible material, such as stainless steel. The barb elements 254 are configured for penetrating and adhering to the tissue when the planar substrate 252 is pressed over the tissue, thereby fixing the neurostimulation lead 14 relative to the tissue. The lengths of the barb elements 254 are longer than the diameter of the neurostimulation lead 14, such that the barb elements 254 can penetrate and adhere to the tissue when the planar substrate 252 is pressed over the neurostimulation lead 14 and tissue.

Alternatively, as illustrated in FIG. 19, the anchoring device 250 further comprises a sleeve 256 having a lumen 258 through which the neurostimulation lead 14 can be secured. The sleeve 256 can be composed as the same material as sleeve 152 discussed above, and the neurostimulation lead 14 can be secured within the lumen 258 of the neurostimulation lead 14 in the same manner as it can be secured within the lumen 154 of the sleeve 152 discussed above. The sleeve 256 is coupled to the other surface of the planar substrate 252 opposite to the surface from which the barb elements 254 extend. In this case, the barb elements 254 may be shorter than the diameter of the neurostimulation lead 14.

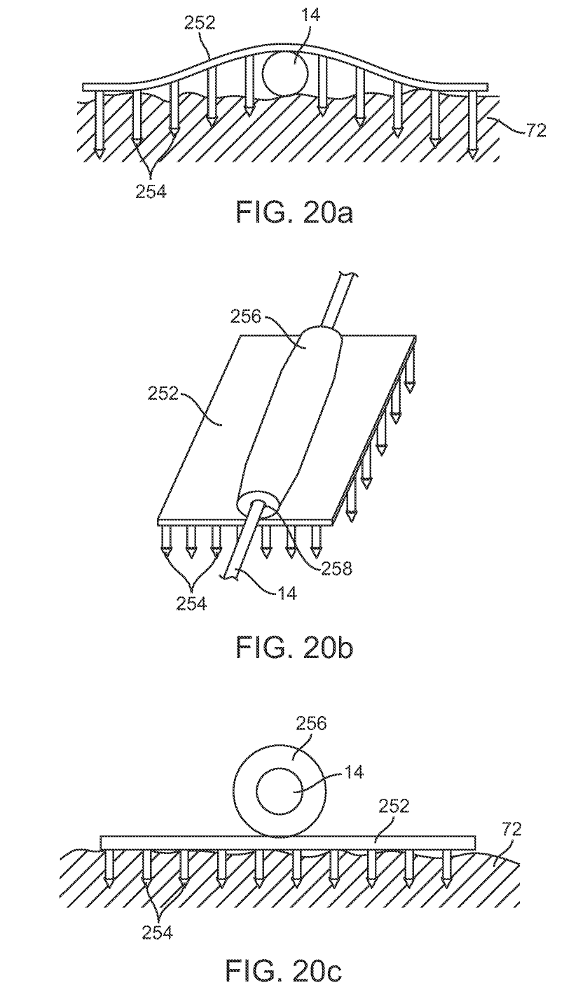

Having described the structure of the anchoring device 250, another exemplary method of fixing the neurostimulation lead 14 relative to solid tissue 72 using the anchoring device 250 will now be described with respect to FIGS. 20a-20c.

In the case where the barb elements 254 are longer than the diameter of the neurostimulation lead 14, the neurostimulation lead 14 is first placed relative to the tissue 72 in the same manner described above with respect to FIG. 5a, after which planar substrate 252, with the barb elements 254 facing down, is pressed down over the neurostimulation lead 14 and tissue 72, such that the barb elements 254 are embedded within the tissue 72 to fix the neurostimulation lead 14 relative to the tissue 72 (FIG. 20a).

In the case where the anchoring device 250 includes the sleeve 256, the neurostimulation lead 14 is introduced through and secured within the lumen 258 of the sleeve 256 (FIG. 20b), and the planar substrate 252, with the neurostimulation lead 14 secured within the lumen 258 of the sleeve 256, is pressed down over the tissue 72, such that the barb elements 254 are embedded within the tissue 72 to fix the neurostimulation lead 14 relative to the tissue 72 (FIG. 20c).

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. An anchoring device for a lead placed on solid tissue, comprising:
a pair of lever arms composed of a biocompatible material, each of the lever arms including a tissue grasping portion and a finger portion;
a hinge coupling the lever arms together between the tissue grasping portion and finger portion of the respective lever arms; and
a biasing mechanism configured for biasing the tissue grasping portions together while biasing the finger portions away from each other to place the tissue grasping portions in a closed state capable of gripping the lead while grasping the solid tissue.

2. The anchoring device of claim 1, wherein the biasing mechanism is a spring mechanism having a spring force allowing the finger portions to be displaced toward each other when a manual force is applied to the finger portions to place the tissue grasping portions in an open state capable of allowing the tissue grasping portions to receive the lead, and allowing the finger portions to be displaced away from each other when the manual force is released from the finger portions.

3. The anchoring device of claim 2, wherein the spring mechanism comprises at least one torsion spring disposed around the hinge.

4. The anchoring device of claim 1, wherein each finger portion of the respective lever arms includes a plurality of teeth that are capable of embedding within the solid tissue when the manual force is released from the finger portions.

5. The anchoring device of claim 1, wherein the solid tissue is fascia.

6. The anchoring device of claim 1, wherein the lead has a diameter in the range of 0.03 inches and 0.07 inches.

7. A method of fixing a lead relative to solid tissue using the anchoring device of claim 2, comprising:
placing the lead relative to the solid tissue;
applying the manual force to the finger portions to place the tissue grasping portions in the open state;
locating the anchoring device in contact with the solid tissue about the lead;
releasing the manual force from the finger portions to place the tissue grasping portions in the closed state, thereby gripping the lead while grasping the solid tissue.

8. The method of claim 7, wherein each finger portion of the respective lever arms includes a plurality of teeth that embed within the solid tissue when the tissue grasping portions grasp the solid tissue.

9. The method of claim 7, wherein the solid tissue is fascia.

10. The method of claim 7, wherein the lead is a neurostimulation lead.

11. The method of claim 7, further comprising inserting the lead into an epidural space.

12. The method of claim 7, further comprising coupling the lead to a neurostimulator.

13. An anchoring device for a lead placed on solid tissue, comprising:
a flange composed of a biocompatible, malleable, material, the flange being pre-curved such it has two opposing sides, a convex outer surface, and a concave inner surface capable of receiving the lead; and
two rows of teeth respectively extending from the two opposing sides of the flange;
wherein the flange is configured for deforming when a manual compressive force is applied to the convex outer surface adjacent the two opposing sides, thereby gripping the received lead and embedding the teeth within the solid tissue.

14. The anchoring device of claim 13, wherein the pre-curved flange is a cylindrical segment.

15. The anchoring device of claim 13, wherein the flange is pre-curved at least 180 degrees.

16. The anchoring device of claim 13, wherein the teeth are pre-curved in the same direction in which the flange is pre-curved.

17. The anchoring device of claim 13, wherein the two rows of teeth are configured for respectively meshing with each other when the flange is deformed.

18. The anchoring device of claim 13, wherein the solid tissue is fascia.

19. The anchoring device of claim 13, wherein the lead has a diameter in the range of 0.03 inches and 0.07 inches.

20. A method of fixing a lead relative to solid tissue using the anchoring device of claim 13, comprising:
placing the lead relative to the solid tissue;
receiving the lead within the pre-curved flange while the anchoring device is located in contact with the solid tissue about the lead; and
applying the manual compressive force to the convex outer surface adjacent the two opposing sides of the pre-curved flange, thereby gripping the lead and embedding the teeth within the solid tissue.

21. The method of claim 20, wherein the solid tissue is fascia.

22. The method of claim 20, wherein the lead is a neurostimulation lead.

23. The method of claim 20, further comprising inserting the lead into an epidural space.

24. The method of claim 20, further comprising coupling the lead to a neurostimulator.

25.-74. (canceled)
